(19) United States
(12) Patent Application Publication
(10) Pub. No.: US 2008/0027756 A1
(43) Pub. Date: Jan. 31, 2008

(54) Systems and methods for identifying and tracking individuals

(76) Inventors: Richard Gabriel, Sarasota, FL (US); Tony N. Frudakis, Bradenton, FL (US); Matthew J. Thomas, Bradenton, FL (US)

Correspondence Address: Lisa A. Haile, J.D., Ph.D.;DLA PIPER US LLP, Suite 1100, 4365 Executive Drive, San Diego, CA, 92121-2133, US

(21) Appl. No.: 11/824,327

(22) Filed: Jun. 29, 2007

Related U.S. Application Data
(60) Provisional application No. 60/817,760, filed on Jun. 30, 2006.

(51) Int. Cl. G06Q 50/00; A61B 19/00

(52) U.S. Cl. 705/2; 604/403

(57) ABSTRACT

Systems and methods for identifying and tracking individuals and samples are disclosed. The system includes a sample container, an individual's identification sample portion coupled to the sample container and means for identifying the individual's identification sample portion. The individual's identification sample portion may be configured to accept fingerprints, human iris scan, human eye color, facial recognition photo and/or DNA. A method is disclosed for using the system for identifying and tracking a sample from an individual. The method includes placing the sample from the individual in a sample container, obtaining and placing an individual's identification sample on an individual's identification sample portion coupled to the sample container, and identifying the individual's identification sample using appropriate instruments to identify the sample.

### RELATED APPLICATION DATA

This application claims benefit of priority under 35 U.S.C. §119(e) to U.S. Provisional Application No. 60/817,760, filed Jun. 30, 2006, which is incorporated by reference in its entirety.

### BACKGROUND OF THE INVENTION

1. Field of the Invention

The present invention relates generally to identifying and tracking individuals, and more particularly, to identifying and tracking individuals using samples from the individuals as their own unique identifier.

2. Background Information

The bulk of modern-day genetic diversity fits most parsimoniously within a 4-population parental model corresponding to the major continents of Europe, Asia, Africa and the Americas. This structure is derived from the expansion of divergent parental populations that were established on each continent after the expansion out of Africa. It is well documented that the majority (80-90%) of the genetic variation among human individuals is inter-individual and only 10-20% of the variation is due to population differences. Further, most populations share alleles and that those alleles that are most frequent in one population are also frequent in others. There are very few classical (blood group, serum protein, and immunological) or DNA genetic markers which are either population-specific or have large frequency differentials among geographically and ethnically defined populations. Despite this apparent lack of unique genetic markers, there are marked physical and physiological differences among human populations that presumably reflect long-term adaptation to unique ecological conditions, random genetic drift, and sex selection. In contemporary populations, these differences are evident both in morphological differences between ethnic groups and in differences in susceptibility and resistance to disease.

Genetic markers that exist in one population and not in others have been referred to as “private”, and such markers can be used to estimate mutation rates. Other descriptions include the term “ideal” (in reference to their utility in individual ancestry estimation) to depict hypothetical genetic marker loci at which different alleles are fixed in different populations. Such variants are also known as “unique alleles,” which variants seem to be found in only one population. The most useful “unique alleles” for forensic analysis are those that also have large differences in allele frequency among populations. The fact that they are totally absent from all other populations does simplify some of the statistical computations and can facilitate more confident parental allele frequency estimates, but is not the primary reason for their utility. These markers have been described by the designation population-specific alleles (PSAs) to identify genetic markers with large allele frequency differentials between populations, but are now referred to as Ancestry Informative Markers (AIMs). For a biallelic marker, the frequency differential (δ) is equal to pₓ-p_{y} which is equal to q_{y}-qₓ, where px and py are the frequencies of one allele in populations X and Y and qₓ and q_{y} are the frequencies of the other. Median δ levels among major ethnic groups range between 15% and 20%, and the vast majority (>95%) of arbitrarily identified biallelic genetic markers have δ<50%.

Every year in the United States there are an estimated 3.9 million clinical trial patients whose samples need to be tracked and validated to ensure that medications and follow up research results of clinically obtained samples from the patients are properly stored and tracked. During these clinical trials, individuals submit to human testing of new diagnostics and drugs, new combinations of approved drugs, or approved drugs in new indications. Data from the trials is then submitted with an application to the FDA requesting permission to sell, distribute and license the technology as an approved medical application. During this process, most of the tracking is limited to:
1. Recording patient information; sometimes taking a patient blood sample, placing it on a DNA storage card and filing it away.
2. Providing the patient with an identification number and assigning a barcode to that patient that corresponds to all necessary patient information, file location etc. according to HIPPA rules and regulations about preserving patient identity.
3. Transporting patient files and patient samples manually from location to location, using the barcode tracking as a means of quality control and quality assurance.
4. Tracking patient samples with other monitoring devices such as radio frequency, magnetic resonance, infrared detection, ion detection, fragment detection, color detection, microchip tagging or any other means of inserting, adding, conjugating or otherwise tagging a patient sample, record or other information.

This system is lacking in three important respects for clinical trial patient management and patient sample tracking, storage and further analysis. These include:
1. Barcode does not insure sample and patient are linked if patient sample is mislabeled or lost.
2. Radio frequency devices, magnetic resonance devices, infrared devices, microchips or any other form of added monitoring device, either internal or external to the patient sample also does not insure sample and patient are linked if patient sample is mislabeled by any such device.
3. No other proof of patient sample-to-patient correspondence exists except the stored blood spot on a card, human tissue sample, fiber with DNA or other patient sample that contains DNA and can be stored by a number of methods, procedures or other such preservations as well known to those in the art of DNA storage and preservation techniques, methods, inventions or literature references.

### SUMMARY OF THE INVENTION

The present invention discloses systems and methods for identifying and tracking individuals and samples therefrom.

In one embodiment, the system includes a sample container, an individual's identification sample portion coupled to the sample container and means for identifying the individual's identification sample portion. The individual's identification sample portion may be, for example, configured to accept a fingerprint, human iris scan, human eye color, facial recognition photo, DNA, or a combination of these biometrics.

In another embodiment, a method is disclosed for using the system for identifying and tracking a sample from an individual. The method includes placing the sample from the individual in a sample container, obtaining and placing an individual's identification sample on an individual's identification sample portion coupled to the sample container, and identifying the individual's identification sample using appropriate instruments to identify the sample.

In one embodiment, a method of identifying and tracking a sample from an individual is disclosed including a user interacting with a graphic interface to enter data associated with the individual and inputting biometric data, where the data includes, but is not limited to, DNA data, physical characteristics, and medical information, or a combination thereof, sending the biometric data to a database comprising authentic biometric data previously obtained from the individual, processing the biometric data to compare the inputted biometric data with corresponding authentic biometric data, and verifying the identity of the individual if the inputted biometric data meets a minimal criteria when compared with the authentic biometric data.

In one aspect, the physical characteristics include a photograph of the individual, a fingerprint of the individual, or an eye scan of the individual.

In another aspect, the method includes placing a DNA sample in a container, attaching a label which annotates the sample based on inputted biometric data, and associating the sample with a data file, where the data file includes biometric data including, but not limited to, physical characteristics and medical information, or a combination thereof.

In another aspect, the threshold for identification for a DNA sample includes contacting the DNA sample with hybridizing oligonucleotides, where the hybridizing nucleotides can detect nucleotide occurrences of single nucleotide polymorphisms (SNPs) of a panel of at least about ten ancestry informative markers (AIMs) indicative of a population structure correlated with the trait, and where the contacting is performed under conditions suitable for detecting the nucleotide occurrences of the AIMs of the test individual by the hybridizing oligonucleotides, and identifying, with a predetermined level of confidence, a population structure that correlates with the nucleotide occurrences of the AIMs in the test individual, wherein the population structure correlates with a trait.

### BRIEF DESCRIPTION OF THE DRAWINGS

FIG. 1 illustrates triangle and tetrahedron plots that are used to visualize the distribution of individual BGA estimates. On a triangle plot, individual ML BGA estimates are plotted using a grid system that helps to display three dimensional space in two dimensions. Points at a vertex represents 100% affiliation with the ancestral population represented at that vertex. Individuals plotting along an axis represent proportional ancestry from each of the two vertices delimiting the axis and have no contribution from the opposite vertex. A line is dropped from each vertex to the opposite base to create the scales against which the ML estimate is projected in order to translate percentages of BGA admixture to positions within the triangle.

FIG. 2a shows global association of autosomal admixture for European, Sub-Saharan African, East Asian, and Native American groups.

FIG. 2b shows a finer map of FIG. 2a.

FIG. 2c shows STRUCTURE with STRs for Sub-Saharan African admixture in Mediterranean and Middle East populations as a function of moving south into North Africa.

FIG. 3 illustrates triangle plots that are used to visualize the distribution of European estimates.

FIG. 4 illustrates triangle plots that are used to visualize the distribution between US Caucasians and continental Europeans.

FIG. 5 illustrates triangle plots that are used to visualize the distribution of African estimates.

FIG. 6 illustrates triangle plots that are used to visualize the distribution of Asian estimates.

FIG. 7 illustrates triangle plots that are used to visualize the distribution of Hispanic estimates.

FIG. 8 illustrates triangle plots that are used to visualize the distribution of Recognized vs. Unrecognized Amerind Tribe estimates.

FIG. 9 shows a plot for percent African ancestry vs. Melanin index in Puerto Ricans.

FIG. 10 shows a return from querying the database for a particular set of individuals.

FIG. 11 shows a DNAPrint analysis of a sample common to several of the rape/murder scenes and determined the donor was a person of 85% sub-Saharan African/15% Native or Indigenous American mix, and more importantly, likely to express a darker skin shade with other overtly African characteristics.

FIG. 12 illustrates the quantification of iris colors using digital photographs and spectrophotomeric software.

FIG. 13 demonstrates the concordance of iris colors among samples of the same multilocus OCA2 genotypes.

FIG. 14 shows an examples of a part of a case report used to communicate an inference or prediction to investigators.

### DETAILED DESCRIPTION

Unless otherwise defined, all technical and scientific terms used herein have the same meaning as commonly understood by one of ordinary skill in the art to which this invention belongs. Although systems, methods and materials similar or equivalent to those described herein can be used in the practice or testing of the present invention, suitable systems, methods and materials are described below. All publications, patent applications, patents, and other references mentioned herein are incorporated by reference in their entirety. In addition, the materials, methods, and examples are illustrative only and not intended to be limiting.

Also, use of the “a” or “an” are employed to describe elements and components of the invention. This is done merely for convenience and to give a general sense of the invention. This description should be read to include one or at least one and the singular also includes the plural unless it is obvious that it is meant otherwise.

Embodiments of systems and methods for identifying and tracking individuals and samples are described herein. In the following description, numerous details are provided, such as the identification of various systems and methods, to provide an understanding of embodiments of the invention. One skilled in the art will recognize, however, that embodiments of the invention can be practiced without one or more of the details, or with other methods, components, materials, etc. For the sake of brevity, well-known instruments, processes, or operations are not shown or described in detail to avoid obscuring aspects of various embodiments of the invention. For example, the present invention may employ various instruments, laboratory equipment, computer hardware, connectivity or use of the Internet, analysis of DNA and barcode systems that include hardware, software, and/or firmware components configured to perform the specified systems and methods.

The present invention concerns identifying and tracking individuals and samples from those individuals. Currently, identifying and tracking of individuals is mainly done with the use of an assigned identification number (such as a medical record number) or barcodes assigned to the individuals. These same numbers or barcodes are then assigned to samples obtained from the individual. Many times the samples are confused or mis-labeled, indicating that sample identification procedures beyond a number or barcode on, e.g., the side of a tube containing a sample needs to be included. The present invention provides a solution to this problem by providing systems and methods for identifying and tracking individuals and samples from those individuals. The present invention may also be used with a barcode system.

One embodiment of the present invention includes a sample container for holding an individual's sample. To identify the sample, the sample container includes an individual's identification sample portion. The term “identification sample portion” as used herein means a structure or device for accepting biometric information or a biometric sample when the biometric is to be used in testing, examination, or study. The individual's identification sample portion uses human identification to link the sample to the individual.

Within the field of human identification are several methods for human identification. The present invention may use one or more of these in a system, which may include, but is not limited to:
1. Fingerprint identification using accepted recording and verification methods and techniques for confirming the identity of an individual;
2. Human iris scan;
3. Human eye color (developed by DNAPrint Genomics, Inc.);
4.Facial recognition (photos and scans of photos, a photo database similar to the photo data base system uses to support DNAWitness for both eye color and facial recognition);
5. Mitochondrial and Y-chromosome DNA testing; and
6. Other DNA testing practices, such as Alu or micro-satellite testing, and including real time PCR, whole genome scan or haplogroup matching, and the like.
7. Determination of the origin of genetic ancestry by using a commercial product such as AncestrybyDNA, EURODNA or other similar or derivative products (developed by DNAPrint Genomics, Inc. or other companies, corporations, individuals, groups of individuals, entities or institutions).
8. Particular use for the determination of categorizing samples, patient results, patient identification, determination of associations between patient samples, relationships, gene structure or any other DNA or RNA related determination for matching, comparing, analyzing, evaluating, predicting or calling out the similarities or dissimilarities between one sample and another.
9. Determining the state of degradation and the combined use of amplifying technologies or other such developed technologies to match samples from one individual to another or earlier samples of the same individual that may have gotten lost, degraded or shipped not in accordance with standard DNA and biological sample handling procedures, resulting in the questionable status of one sample compared to another.
10. Validating and comparing samples of tissue, blood, organs, cells or preserved, dried or desiccated tissues or samples with known and verifiable human features as recorded by any other electronic, mechanical, chemical or biological means such as photos, eye scans, fingerprints, hair color, facial recognition or other phenotypic markers such as P450 enzymes, drug metabolism rates, genetic variants and particular markers, proteins, enzymes or other significant biological and cellular markers that are determined to be unique in whole or in combination with other variants and confirmed by analysis and comparison.

The present invention utilizes an individual's own unique identifier, phenotypic marker or biometric data, such as those listed above, and attaches or couples the unique identifier to a sample for identification and tracking. A database system may be used that links all patient information to a bar code, fingerprint, eye color (and eye color markers), iris scan, photo of individual and all laboratory results. The database system may store patient information in an Ancestry Database format.

In one embodiment, the database links all patient information to eye color. Polymorphism of the OCA2 gene almost certainly underlies the previous assignment of the brown/blue eye (BEY2/EYCL3, MIM227220) and brown hair (HCL3, MIM601800) loci to chromosome 15q, with two OCA2 coding region variants Arg305Trp and Arg419Gln associated with non-blue eye colors. These variants can be used as a biometric identifier. For example, Table 1 shows the accuracy of correctly identifying individuals using OCA2 variants.

TABLE 1
Determination of sample identity using OCA2 variants.

| Sample | Results: | | | Using: | OCA2- | OCA2- |
|---|---|---|---|---|---|---|
| (n) | Correct | Incorrect | Accuracy | OCA2-A | B | C |
| 100 | 76 | 20 | 79% | yes | no | no |
| 596 | 132 | 11 | 92% | yes | yes | yes |
| 596 | 136 | 7 | 95% | yes | yes | yes |

A DNA ‘bar code’ tracking system may also be utilized that allows researchers to send samples worldwide and link back to the original patient data information (this system may or may not include personal information, such as information that would be in violation of HIPPA rules and regulations). The present system may provide an information sharing environment for researchers that allows them to track samples or locate samples as well as establish the absolute identity of samples and ties those samples to a particular patient. Validation and control of machine operations may communicate via internet hookup, telephone line, wireless communication, and the like.

It should be appreciated that the individual's unique identifier or biometric data may be obtained and/or analyzed by any number of known instruments or devices that include hardware, software, and/or firmware components configured to perform the specified operations and methods. There are numerous instruments that are known that can measure or determine any or all combinations of the above for identification. The present invention may use any instrument on which any or all combinations of the above can be used and combined. In one embodiment, the system of the present invention includes laboratory equipment, computer hardware, internet access and the analysis of DNA, matched to the patient barcode or patient number.

One suitable instrument is the Vidiera™ NsD Nucleic Sample Detection machine from Beckman Coulter. This machine can be augmented by any machine, e.g., using capillary electrophoresis, real time PCR or any other method of determining identity of a DNA sample.

One suitable instrument for fingerprint identification is a fingerprint machine, such as the M2-S fingerprint reader from M2SYS Technology (M2SYS Accelerated Biometric, Atlanta, Ga.). The fingerprint machine includes a scanning area that captures a fingerprint and sends it to a database system or recordation equipment to couple with the sample.

The present invention may employ apparatus and processes similar to those used in CODIS (COmbined DNA Index System) to track criminals. The equipment and methods used in CODIS are known and are not disclosed in detail here. CODIS are an electronic database of DNA profiles that are generated from convicted offenders and/or from crime scene evidence. CODIS enable comparison and data sharing amongst authorized laboratories using an encrypted secure format. CODIS was developed by the U.S. Department of Justice and the FBI and provided at no cost to law enforcement forensic laboratories worldwide. The CODIS system does not ‘track’ samples for a convicted felon.

In one embodiment, the present invention adds a CODIS test to a procedure of accepting and tracking samples. The CODIS information may be placed in the individual's identification sample portion coupled to the sample. The test determines the Short Tandem Repeat (STR) of a human DNA sample and uses the 16 loci as a means of identifying and confirming that the sample that was sent is the sample that was received, or linking the barcode to on STR chromatogram.

In another embodiment, the present invention may use Ancestry Informative Markers (AIMs) and/or other DNA markers that include but are not limited to mitochondrial testing, STR testing, Alu testing, Y-chromosome, whole genome, partial genome as well as any combination of these tests and others to verify and confirm the identity of an individual or an individual's sample as it is evaluated, transported or changed in any way during the clinical development, discovery or research phase of diagnostic or drug development process. AIMs are the subset of genetic markers that are different in allele frequencies across the populations of the world. Most polymorphism is shared among all populations and for most loci the most common allele is the same in each population. An ancestry informative marker is a genetic marker that occurs mostly in particular founder population sets but may also be found in varying levels across all or some of the populations found in different parts of the world. AIMS are described further in U.S. Patent Application Nos: US2007/0037182 and US2004/0229231, the contents of which are incorporated by reference in their entirety.

In another embodiment, the present invention may link BioGeographical Ancestry, Mitochondrial DNA, Y-chromosome DNA, STR analysis to a barcode and/or patient identification number along with a scan of the human iris, an eye picture, a picture of the individual and/or a finger print or any other form of identification that may be used to perform a quick check of patient identity and compliance to treatment programs.

The benefit to hospitals, medical centers and government agencies of the present invention is the ability to track not only individuals and patients, but also samples from the individuals and patients, and to track the absolute identity of the samples as well as the integrity of the samples. The system disclosed may also be used for forensic applications as well in tracking individuals using DNA technologies across the globe.

In another embodiment, the present invention may include a system having an instrument, an identity test and a BioGeographical Ancestry (BGA) test that may be used in a clinical trial patient monitoring program. The combination of equipment, test supplies, software, hardware, database management and mining tools will not only ensure the sample and patient identity; it will establish a baseline for BGA, tied to the self reported information. The combined technology will also help establish a powerful database of clinical and patient results on a strictly confidential basis, preserving HIPPA rules and regulations and should only be employed with patients on a voluntary basis. In addition to patient sample tracking of human tissues for experimentation and study, the system may act as a ‘stability’ monitoring device, ensuring that all samples tested are in compliance with current Good Laboratory Practices (‘cGLP’) and that the information is in compliance with filings for the FDA either under a 510K or other drug applications, i.e., Investigational New Drug Application (IND), Amended New Drug Application (ANDA) or New Drug Application (NDA). The system offers patient verification, patient sample tracking and database storage of potentially vital information. The system will allow tracking of patient samples accurately, and should a treatment protocol change be required, assure researchers that the sample tested belongs to the patient being treated, thereby, preventing overdose or mis-dose situations that could be fatal. The system may also help eliminate or reduce possible ‘professional patients’ and help broaden the mix of patients with diversified BGA's. Additionally, should an investigator determine that a certain series of markers are vital to predicting a patient response to a particular therapy, the instrument, operating under current ASR (analyte-specific reagents) classification can be used as a platform for monitoring those patients with the newly developed test methods that can help physicians more closely monitor the patient's progress through treatment and post treatment. Also, the system can be used to validate and authenticate use new ‘genotype’ tests on a particular patient. If genotyping tests are being performed, the system may act as a storage locker for test results and comparative information resulting from the use of BGA and other tests added to the system to help determine if the genotype test is performing across a wide swath of the human population. In most genotype related tests, a comparison against known inherited markers helps researchers identify potential problems or hotspots on the DNA that may or may not contribute to the ability of the diagnostic to predict patient response.

The invention described herein presents significant benefits that would be apparent to one of ordinary skill in the art. While at least one embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing the exemplary embodiment or exemplary embodiments.

### EXAMPLES

Genetic Ancestry

A battery of 176 AIMs was selected from the human genome based on their information content with respect to the 4-population model, attempting to keep a balance in content for each of the possible pair-wise population comparisons. Table 2 shows some of the AIMs in the panel—their chromosomal location, and δ values for three different population comparisons (AF-sub-Saharan African, EU-European, NA-Indigenous American).

TABLE 2
Pair-wise population comparisons

| MARKER | LOCATION | Mb | AF/EU | AF/NA | EU/NA |
|---|---|---|---|---|---|
| MD575 | 1p34.3 | ˜42 | 0.130 | 0.417 | 0.546 |
| MD187 | 1p32 | ˜50.2 | 0.370 | 0.440 | 0.070 |
| FY-NULL | 1q23.2 | ˜181 | 0.999 | 0.999 | 0.000 |
| AT3 | 1q25.1 | ˜196 | 0.575 | 0.777 | 0.202 |
| F138 | 1q31.3 | ˜220 | 0.641 | 0.674 | 0.033 |
| TSC11020554 | 1q32.1 | ˜234.5 | 0.441 | 0.303 | 0.744 |
| WI-11392 | 1q42.2 | ˜269.5 | 0.444 | 0.256 | 0.188 |
| WI-16857 | 2p16.1 | ˜56.2 | 0.536 | 0.548 | 0.012 |
| WI-11153 | 3p12.1 | ˜95.0 | 0.652 | 0.022 | 0.629 |
| GC*1E | 4q13.3 | ˜75.7 | 0.697 | 0.530 | 0.166 |
| GC*1S | 4q13.3 | ˜75.7 | 0.538 | 0.478 | 0.060 |
| MID-52 | 4q24 | ˜110.7 | 0.186 | 0.500 | 0.687 |
| SGC30610 | 5q11.2 | ˜61.5 | 0.146 | 0.281 | 0.427 |
| SGC30055 | 5q22.1 | ˜124.7 | 0.457 | 0.675 | 0.218 |
| WI-17163 | 5q33.1 | ˜173.9 | 0.120 | 0.641 | 0.521 |
| WI-9231 | 7p22.3 | ˜1.2 | 0.017 | 0.387 | 0.370 |
| WI-4019 | 7q21.3 | ˜100 | 0.124 | 0.173 | 0.296 |
| CYP3A4 | 7q22.1 | ˜101.9 | 0.761 | 0.755 | 0.006 |
| LPL | 8p21.3 | ˜22.3 | 0.479 | 0.521 | 0.042 |
| CRH | 8q13.2 | ˜73.2 | 0.609 | 0.655 | 0.046 |
| WI11909 | 9q21.31 | ˜81.0 | 0.075 | 0.587 | 0.663 |
| D11S429 | 11q13.3 | ˜70.4 | 0.429 | 0.054 | 0.376 |
| TYR | 11q14.3 | ˜95.4 | 0.444 | 0.055 | 0.389 |
| DRD2TaqI”D” | 11q23.2 | ˜125.0 | 0.535 | 0.046 | 0.582 |
| DRD2-Bcl I | 11q23.2 | ˜125.0 | 0.080 | 0.565 | 0.485 |
| APOA1 | 11q23.3 | ˜128.9 | 0.505 | 0.555 | 0.050 |
| GNB3 | 12p13.31 | ˜7.2 | 0.463 | 0.430 | 0.033 |
| RB1 | 13q14.2 | ˜47.4 | 0.611 | 0.711 | 0.100 |
| OCA2 | 15q12 | ˜24.0 | 0.631 | 0.369 | 0.263 |
| WI-14319 | 15q14 | ˜30.0 | 0.185 | 0.310 | 0.494 |
| CYP19 | 15q21.2 | ˜47.6 | 0.045 | 0.379 | 0.423 |
| PV92 | 16q23.3 | ˜96.5 | 0.073 | 0.551 | 0.624 |
| MC1R314 | 16q24.3 | ˜103.8 | 0.350 | 0.441 | 0.090 |
| WI-14867 | 17p13.2 | ˜3.5 | 0.448 | 0.404 | 0.045 |
| WI-7423 | 17p13.1 | ˜8.2 | 0.476 | 0.074 | 0.402 |
| Sb19.3 | 19p13.11 | ˜27.0 | 0.488 | 0.236 | 0.253 |
| CKM | 19q13.2 | ˜55.8 | 0.150 | 0.694 | 0.545 |
| MID-154 | 20q11.23 | ˜34 | 0.444 | 0.368 | 0.076 |
| MID-93 | 22q13.2 | ˜38.6 | 0.554 | 0.179 | 0.733 |

The markers proposed for addition to BIOCHIP herein were discovered from a high-throughput screen of 27,000 SNPs part of the NCBI:dbSNP database, with allele frequencies from previously published data (Akey et al., Genome Res (2002) 12:1805-1814) available at the SNP consortium web site. Initial selection criterion was based on allele frequency difference (δ) between three populations (European American, African American, and East Asian) and markers with δ>0.4 between any two groups were chosen. These candidate AIMs were genotyped following the protocols described elsewhere (Frudakis et al., J Forensic Sci (2003) 48:771-782), in the parental AF, EU, EA and IA samples. The first pass focused on 400 candidate AIMs with the largest δ values from which these 71 validated AIMs were selected based on a minor allele frequency greater than 0.01 and of δ>0.4 for at least one of the group pairs. Additional markers were selected to enrich the existing panel for markers with greater power to distinguish between non-African (EU, IA and EA) populations. Estimates of Locus-Specific Branch Length (LSBL; Shriver et al., Hum Genomics (2004) 112:387-399) were used to screen the previously mentioned data set and a second data set posted by Affymetrix on the same three populations (Kennedy et al., Nat Biotechnology (2003) 21:1233-1237). LSBL makes it possible to geometrically isolate the divergence between populations at a given locus and thus identify loci with high divergence times for any one population compared to two other populations. Departures from independence in allelic state within and between loci were tested using the MLD exact test (Zaykin et al., Genetica (1995) 96:169-178). The 176 AIMs part of the final panel are distributed across all autosomal chromosomes with an average of 3 AIMs/chromosome.

Selection of Parental Samples and Nomenclature

As discussed already, previous studies have suggested the bulk of modern-day genetic diversity fits most parsimoniously within a 4-population parental model corresponding to the major continents of Europe, Asia, Africa and the Americas (Rosenberg et al., Science (2002) 298:2381-2385); Shriver et al., 2004, supra). This structure is derived from the expansion of divergent parental populations that were established on each continent after the expansion out of Africa starting 50KYA. We identified samples from these continents that were expected to be good (unadmixed) representatives of these parental populations, and we tested each for their suitability as such using the program STRUCTURE. These included 271 relatively homogeneous descendants from these four parental population groups, West Africans (AF), Europeans/European Americans (individuals self reporting as Caucasians) (EU), East Asians (EA) and Indigenous Americans (IA), were genotyped for establishing ancestral allele frequencies for selected AIMs: 70 AF samples from Nigeria, Sierra Leone and Central African Republic (Parra et al., Am J Phys Anthropol (2001) 114:18-29), 68 IA samples comprising of Mixtec and Nahua individuals from Guerrero, Mexico (Bonilla et al., Am J Phys Anthropol (2005) 128:861-869), 67 EA samples including 10 Chinese and 10 Japanese samples from the Coriell cell repository (purchased from Coriell Institute, Camden, N.J.) and an additional 47 first/second generation Asian Americans from different US locales, and 66 EU samples from various US locales including individuals who self report as “Caucasians”. Using Principal Components Analysis, parental samples cluster as expected into groups corresponding to the 4 continents and each of the samples selected as representatives of a given parental population clustered together. Significant levels (e.g., >15%) of inter-group affiliation for a few of the parental samples indicated admixture or elements of structure superfluous to the 4-population model so these samples were discarded from the analysis. Eliminating samples with less than 85% affiliation with their continental group, the final parental sample used for estimating allele frequencies consisted of 66 EU, 67 IA, 68 EA and 70 AF individuals.

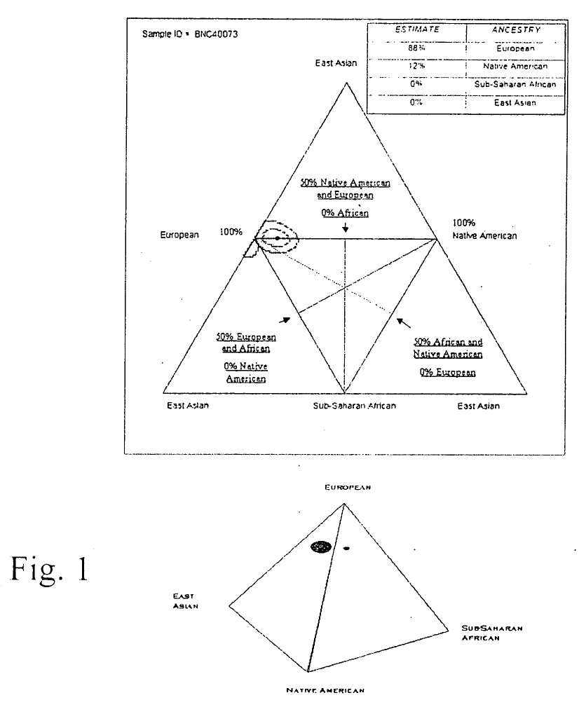

The choice of terminology for these parental populations needs some discussion here. Our social notions of “race” are highly correlated with genetic ancestry but the correlation is not perfect. The elements of population structure we measure with a genetic ancestry panel are therefore entities that most individuals do not necessarily understand or appreciate. For example, the average Hispanic in FIG. 1 probably does not appreciate the lesson shown by this figure, or realize where on the axis of Indigenous American—European admixture he or she falls. They identify as a Hispanic. Many people of Eurasia and South Asia show “European” ancestry, and some of Eurasia with no history of American Indian relatives show low levels of “Indigenous American” admixture. At first glance, these results might seem to represent statistical error, until we realize that the admixture is found systematically in populations from this part of the world and that on the level of the population, the finding is highly significant. The fact that results like this seem difficult to explain is because the elements of structure we measure are not confined to specific geographical regions and modern-day populations but spread throughout the world as a function of the history of each parental population. The terminology we use imperfectly relates to this spread because it is based on modern-day geopolitical terms (like European—Europe is a continent, not a people). The choice of nomenclature for the four continental elements of population structure is somewhat arbitrary; we follow conventions established by others before us and use modern-geographic descriptors based on the locations from which the samples were derived (sub-Saharan African, European, East Asian and Indigenous American). Our 176 Ancestry Informative Markers (AIMs) chosen from the genome based on their information for a continental, 4-population model, which lends itself to use of the terms “European” (genetic ancestry shared among Europeans, Middle Eastern and to a lower extent South Asians), “sub-Saharan African”, “East Asian” and “Indigenous American” (genetic ancestry shared among American Indians, Latin and South American Indians and certain Central Asian populations). The names of these parental populations were chosen to describe extant elements of genetic structure and are arbitrary in that they are reflective of modern population distributions—not necessarily the distributions of the original parental populations 15,000-50,000 years ago. We identified samples from these continents that were expected to be good (unadmixed) representatives of these parental populations, and we tested each for their suitability as such using the program STRUCTURE. Parental samples clustered as expected into groups corresponding to the 4 continents (FIG. 1). Each of the samples selected as representatives of a given parental population clustered together. Significant levels (e.g. >15%) of inter-group affiliation for a few of the parental samples indicated admixture or elements of structure superfluous to the 4-population model so these samples were discarded from the analysis. Although this nomenclature is rational in light of the paleoarchaeological, linguistic and more recent ethnogeographic history of the human species—as well as common sense—it is important to point out that these choices are for convenience rather than meant to strictly delimit the geographical ranges within which the parental populations ultimately derived. For example, the parental population that contributed to the modern-day “European” diaspora was not necessarily confined to “Europe” (whatever longitude and latitude Europe begins and ends). “Indigenous American” in our terminology could probably be better described (as we have seen from the global distributions of ancestry) as American/Central Asian or with a roman numeral IV etc. The term Indigenous American implies a geographical boundary within which the element of population structure is confined, which is clearly inconsistent in all probability with the highly complex nature of how and when our ancestors arose, spread and how their modern-day diaspora are distributed globally.

Admixture Estimation

To infer the ancestry of the alleles at the locus from the marker genotype, we require the ancestry-specific allele frequencies—the conditional probability of each allelic state given the ancestry of the allele (West African or European, for example). Let us imagine the total population of alleles at any locus in the admixed population as made up of two subpopulations: alleles of African ancestry and alleles of European ancestry. As long as the ancestry-specific allele frequencies are correctly specified for the admixed population under study, we can apply Bayes's theorem to invert these conditional probabilities and calculate the posterior distribution of ancestry at the locus (0, 1 or 2 alleles of African ancestry) for each individual under study. If the information conveyed by typing a single marker is not sufficient to assign the ancestry of each allele at the marker locus to one of the two founding populations, markers can be combined in a multipoint analysis to estimate ancestry at adjacent loci. Simulation studies show that with enough markers, a high proportion of information about ancestry at each locus can be extracted even though no single marker is fully informative for ancestry (McKeigue P, Am J Hum Genet (1998) 63:241-251).

The estimation of multi-point admixture (more than 2 groups) is best explained using an analogy. Estimating individual admixture proportions using the Bernstein formula in Bernstein 1932 is most easily accomplished with likelihood functions. The easiest way to explain how the likelihood function works to a novice is through analogy. Consider a hypothetical town with 4 clothing stores. Each store specializes in clothes of a different color—one sells mainly blue clothes, another mainly red cloths, and the others yellow and green. Each of the stores is not exclusive to a color—the red store also sells yellow, green and blue items, but assume that 95% of the items in the red store are red and so on for the other stores. Assume each person in the town shops exclusively among these 4 stores. Walking the streets of this town, we can count the number of differently colored clothing items worn by each person and form a probability statement to express their most likely shopping habits—which stores they spend the most time in, and the proportion of time spent in each store. The larger the number of clothing items we consider, the more accurate is our probability statement. This is analogous to the relationship between the number of AIMs and the quality of the ancestry inference with individual ancestry estimations—and why a large number are needed to do a good job.

Allele frequencies at marker loci are estimated from the relatively unadmixed set of individuals representing each ancestral population. Subsequently, these allele frequencies are used in a maximum likelihood (ML) method (Bernstein F., Die geographische Verteilung der Blutgruppen und ihre anthropologische Bedeutung, 1931, pp. 227-243, Instituto Poligraphico Dello Sato, Rome) for estimating individual BGA proportions. The ML method is described elsewhere (Hanis et al., Am j Phys Anthropol (1986) 70:433-441) and is used to infer individual ancestry under dihybrid and trihybrid models of admixture (Bonilla et al., Hum Genet (2004) 115:57-68; Shriver et al., 2004, supra; Shriver et al., Am J Hum Genet (1997) 60:957-964). We and our colleagues have written software (IAE3CI written by Carrie Pfaff, Mark Shriver, Visu Ponnuswammy) for ML calculations. On an individual level, admixture proportion is inferred from calculating the probability of observing an allele given the allele frequency for that allele in the ancestral populations, which have contributed to the individual under study. Briefly, admixture proportions for an individual are first estimated at a single locus and a corresponding likelihood score assigned to it. Log likelihood scores over multiple loci are summed to determine the likelihood that an individual with observed multilocus genotype has a certain proportionality of ancestry distributed among the various populations. In practice, all possible types of admixture proportions given the multilocus genotypes are defined and corresponding likelihood scores for each proportion are computed. The admixture proportion that maximizes the combined likelihood function across all loci is used as the point estimate of individual BGA. For accommodating the 4 population model, all possible 3-way admixture models were computed using the algorithm previously described (Bonilla et al., 2004, supra; Shriver et al., 2004, supra; Shriver et al., 1997, supra) and the 3-way model with the highest likelihood was chosen. When the second best 3-way model fell within 1 log of the best 3-way model, a 4-population ML calculation method was used and the results presented as a function of 4 percentages. This grid method was used for all analyses unless mentioned otherwise and is henceforth referred to as the ML test. It is assumed that a low level of likelihood difference among different 3-model likelihoods is an indication that affiliation with all 4 populations exists for the sample. The effect of heterogeneity in biasing the estimates of the genetic contributions to an admixed population can be reduced by selecting markers showing homogeneity within the main parental populations (Europeans and Africans). In this way, the problem of contribution of different geographical areas to the parental populations is minimized, reducing the bias in admixture estimates. We have implemented this strategy in our previous admixture studies (Parra et al., Am J Hum Genet (1998) 63:1839-1851; Parra et al. 2001, supra; Pfaff et al., Am J Hum Genet (2001) 68:198-207). We have systematically analyzed potentially informative markers in different European and African populations. As an example, currently, to test for heterogeneity within Africa, we genotype each potentially informative marker in samples from five African populations, two from Nigeria, two from Sierra Leone and one from Central African republic, and the markers showing significant heterogeneity are excluded from the analysis. In addition to this strategy, it is important to note that there are statistical methods to test for misspecification of parental frequencies. One such method has been described in one of the papers our collaborator has jointly published with Dr. Paul McKeigue (McKeigue et al., Ann Hum Genet (2000) 64(Pt. 2):171-178).

Triangle and tetrahedron plots are used to visualize the distribution of individual BGA estimates. On a triangle plot, individual ML BGA estimates are plotted using a grid system that helps to display three dimensional space in two dimensions (FIG. 1). Points at a vertex represents 100% affiliation with the ancestral population represented at that vertex. Individuals plotting along an axis represent proportional ancestry from each of the two vertices delimiting the axis and have no contribution from the opposite vertex. A line is dropped from each vertex to the opposite base to create the scales against which the ML estimate is projected in order to translate percentages of BGA admixture to positions within the triangle. For representing all 4 populations a composite tetrahedron of 4 smaller equilateral triangles or sub-triangles, representing all 4 possible 3-way population models are used. Any point within the composite triangle represents proportional percentages for three groups at a time. The tetrahedron allows all possible groups of three to be presented in a 2-dimensional space and folding the composite tetrahedron along each shared triangle base produces a 3-dimensional pyramid within which the 4-dimensional likelihood space can be visualized (bottom, FIG. 1). Two, 5 and 10-fold confidence areas and volumes are determined in the same way and defined by sampling the likelihood space for each individual.

Global Apportionment of Genetic Ancestry

Using the 176 AIMs and corresponding ancestral allele frequencies, we have previously surveyed the global apportionment of genetic diversity with respect to the 4-population model among several sub-populations throughout the world. Table 3 lists the populations studied.

TABLE 3
Ethnic populations surveyed with 176 AIM Individual Genetic Ancestry Admixture
(IGAA) panel with average admixture results (SD).

| Population Group | | | | |
|---|---|---|---|---|
| (N) | EU | AF | EA | IA |
| European American | 0.905 (0.1) | 0.03 (0.058) | 0.028 (0.049) | 0.038 (0.061) |
| (207) | | | | |
| African American | 0.143 (0.133) | 0.796 (0.14) | 0.028 (0.06) | 0.033 (0.051) |
| (136) | | | | |
| North African (7) | 0.774 (0.58) | 0.015 (0.073) | 0.056 (0.054) | 0.02 (0.034) |
| North European (10) | 0.97 (0.036) | 0.01 (0.021) | 0.019 (0.03) | 0.04 (0.014) |
| Irish (17) | 0.964 (0.043) | 0.07 (0.021) | 0.012 (0.027) | 0.017 (0.041) |
| Icelandic (12) | 0.938 (0.0055) | 0.012 (0.022) | 0.008 (0.014) | 0.043 (0.05) |
| Greek (18) | 0.904 (0.04) | 0.048 (0.042) | 0.017 (0.053) | 0.047 (0.048) |
| Iberians (9) | 0.788 (0.21) | 0.066 (0.071) | 0.04 (0.076) | 0.107 (0.167) |
| (ES&Port) | | | | |
| Basque (10) | 0.93 (0.052) | 0.023 (0.036) | 0.08 (0.025) | 0.039 (0.041) |
| Italian (12) | 0.868 (0.089) | 0.032 (0.048) | 0.027 (0.055) | 0.073 (0.059) |
| Turkish (40) | 0.853 (0.054) | 0.023 (0.032) | 0.073 (0.067) | 0.051 (0.06) |
| Ashkenazi Jews (10) | 0.868 (0.058) | 0.047 (0.039) | 0.02 (0.049) | 0.066 (0.036) |
| Middle East v1 (9) | 0.881 (0.097) | 0.028 (0.056) | 0.048 (0.073) | 0.042 (0.051) |
| Middle East v2 (11) | 0.822 (0.11) | 0.108 (0.089) | 0.045 (0.057) | 0.026 (0.063) |
| South Asian Indian | 0.589 (0.089) | 0.051 (0.047) | 0.269 (0.107) | 0.031 (0.088) |
| (56) | | | | |
| Chinese (10) | 0.070 (0.09) | 0 | 0.98 (0.024) | 0.013 (0.025) |
| Japanese (10) | 0.011 (0.016) | 0.04 (0.018) | 0.953 (0.042) | 0.032 (0.042) |
| Atayal (10) | 0.05 (0.016) | 0 | 0.976 (0.042) | 0.019 (0.042) |
| South East Asian | 0.08 (0.111) | 0.036 (0.073) | 0.822 (0.148) | 0.063 (0.067) |
| (11) | | | | |
| Pacific Islander (7) | 0.247 (0.16) | 0.037 (0.045) | 0.506 (0.213) | 0.21 (0.117) |
| American Indian | 0.419 (0.358) | 0.037 (0.124) | 0.067 (0.086) | 0.476 (0.338) |
| (223)* | | | | |
| American Indian | 0.286 (0.276) | 0.022 (0.059) | 0.082 (0.092) | 0.611 (0.27) |
| (170)** | | | | |
| Mexican (60) | 0.432 (0.193) | 0.056 (0.072) | 0.044 (0.093) | 0.468 (0.181) |

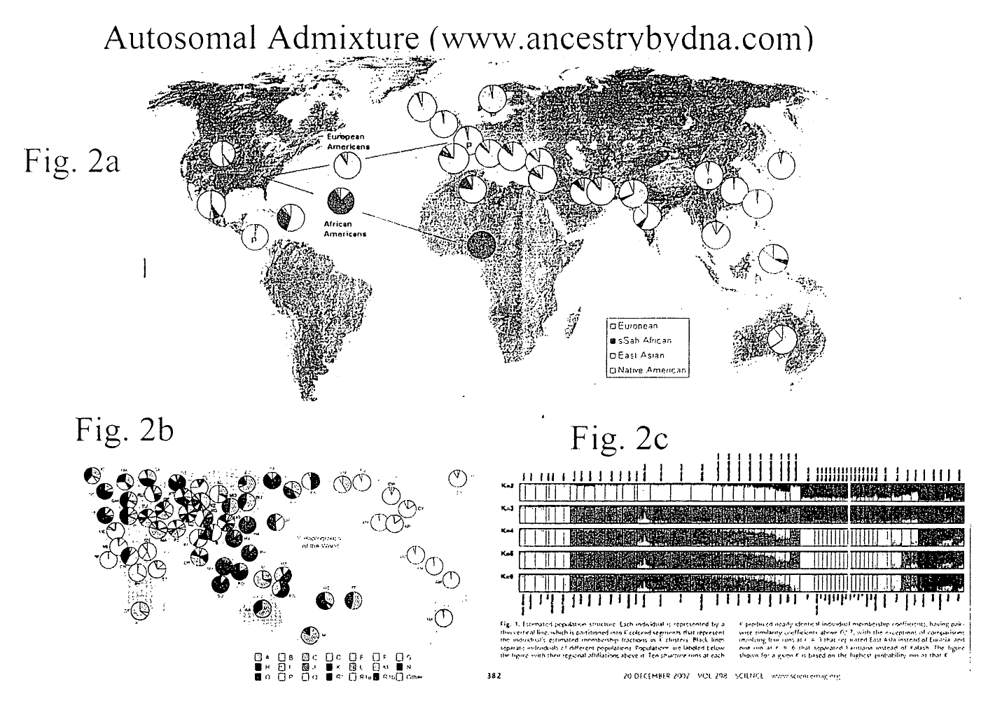

In terms of admixture averages, populations from Europe, the Middle East and South Asia showed high “European” genetic ancestry with increasing “admixture” moving from Northwestern Europe to South Asia (FIG. 2a). Sub-Saharan African admixture was notable in populations along the Mediterranean and Middle East, increasing in fraction farther south into North Africa, where in agreement with results obtained by others using STRUCTURE with STRs (FIG. 2c; Rosenberg et al., 2002, supra), the level was substantial. This pattern was reminiscent of the distribution of the E Y-chromosome haplogroup. Significant East Asian admixture was observed for the South Asian populations reminiscent of results presented previously by others (Chakraborty R., Yearbook Phys Anthropol (1986) 29:1). In East and Southeast Asia, East Asian ancestry dominated with increasing “admixture” outside of China in the southeast. Aboriginal Australians registered with predominant “European” ancestry with our 4-population model and traces of Indigenous American ancestry were found in Southeastern Europe, South Asia, the Pacific Islands and Australia.

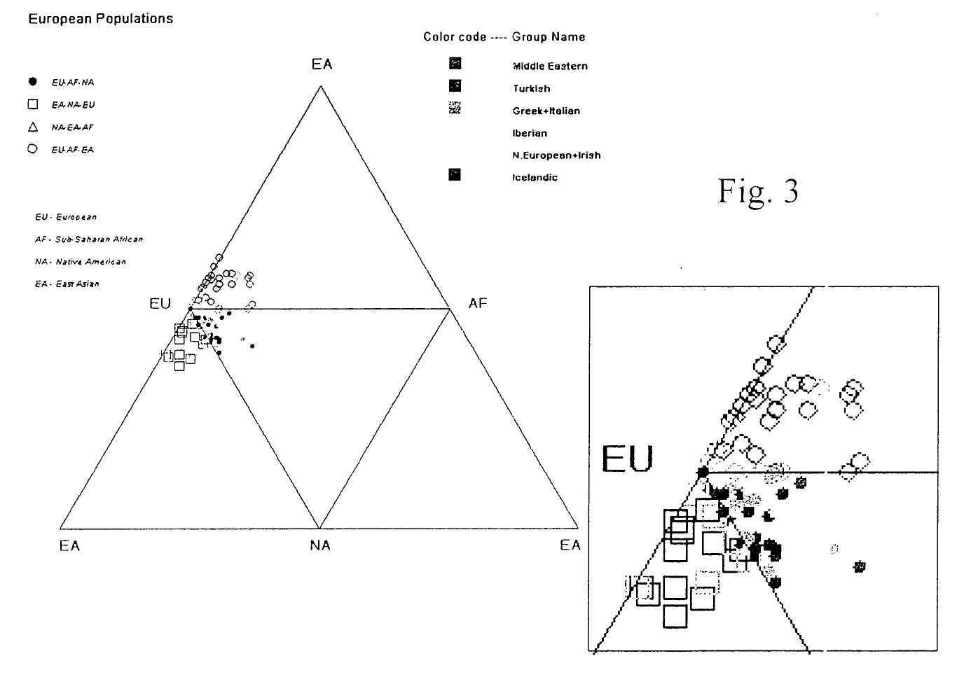

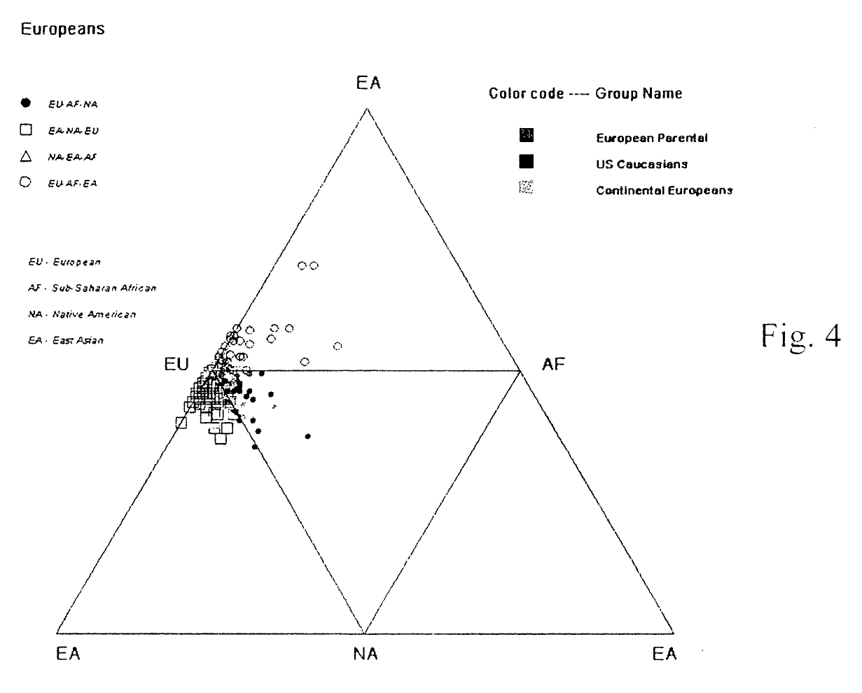

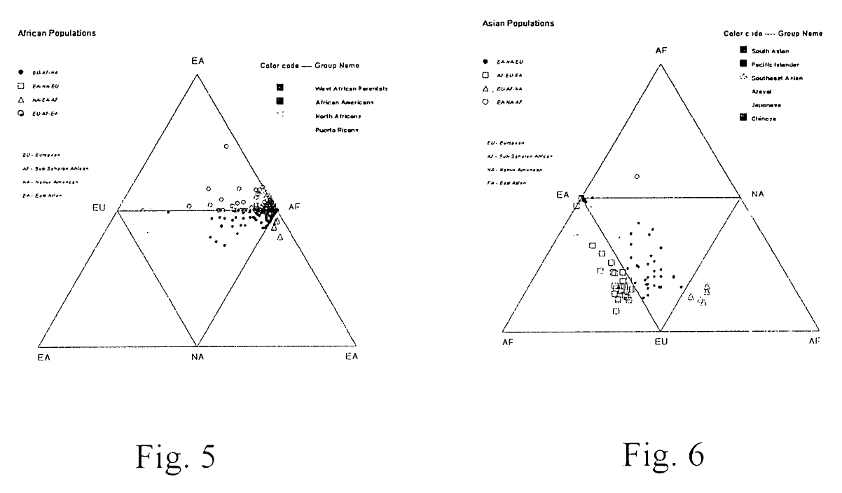

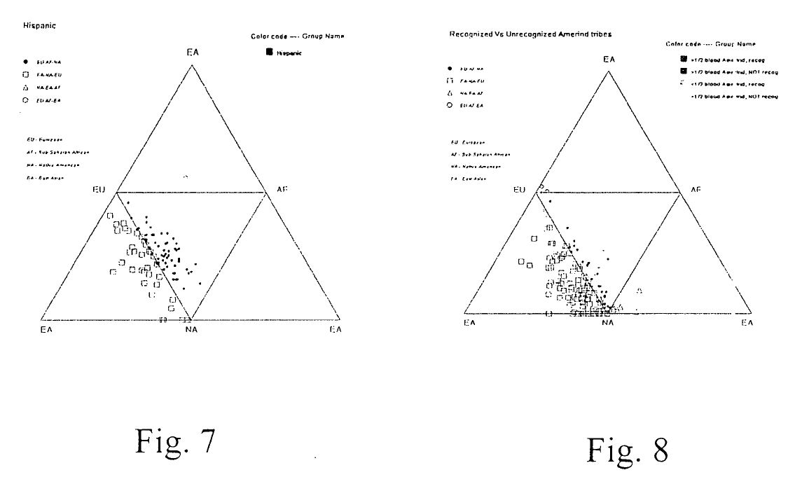

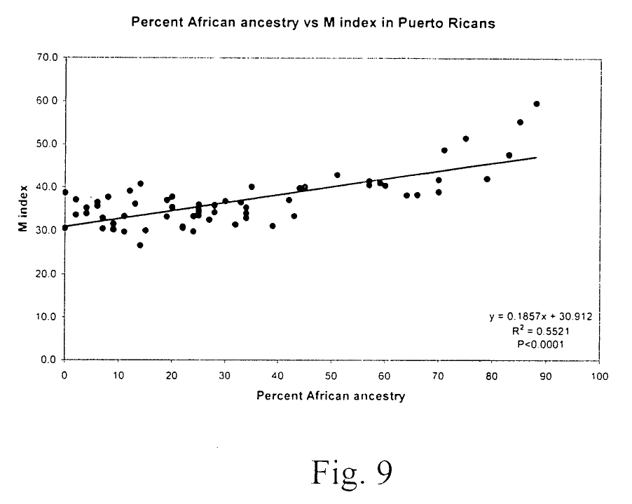

We have also used the 176 AIM panel and DNAPrint admixture program to study intra-population distributions of admixture. While population averages may be useful for reconstructing certain historical interactions between parental ancestry groups, the triangle plots allow for an appreciation of variability and structure within populations. Among European and Middle Eastern populations, variability in non-European “admixture” appears for the most part to be independent of population membership (FIG. 3). That is, there appears to be a clustering of samples to subpopulation groups. For example, while the average non-European admixture for Turkish populations is greater than the average non-European ancestry for Northern Europeans, little overlap between them is observed. Populations from geographical locations intermediate to Northern and Southeastern Europe, such as the Greeks and Italians, cluster to coordinates in the triangle plot intermediate to populations derived from these locations (Northern Europeans and Turks or Middle Eastern populations). Little difference was noted among US Caucasians and continental Europeans (FIG. 4). Among populations of self-described African ancestry, greater non-sub-Saharan African admixture is observed for populations that have historically mixed with others, such as African Americans and Puerto Ricans (FIG. 5). Variation in admixture within these latter two populations was relatively high compared with that observed for parental Africans. The average level of European admixture for African Americans was about 20%, which is similar to results obtained by other authors using other statistical methods (Chakraborty R, 1986, supra). Admixture was exceptionally low among the Chinese, Japanese and Atayal, but significant for Southeast and South Asian populations as well as Pacific Islanders (FIG. 6). Again, there was a notable clustering of samples to population groups. Hispanics show tremendous variation in Indigenous American vs. European admixture, which is belied by the population average of approximately 50%:50% mix (FIG. 7). FIG. 8 shows various populations of self-reported Indigenous American ancestry. Individuals residing on US government recognized American Indian reservations who claim to be of more than half-blood American Indian generally show high Indigenous American ancestry (red symbols, FIG. 8), whereas individuals from those same reservations who claim to be of half blood or less American Indian ancestry generally show lower but significant Indigenous American admixture (green symbols, FIG. 8). In contrast, little Indigenous American admixture was detected for two Caucasian populations of urban individuals not living on federally recognized reservations but claiming American Indian ancestry. All of these results taken together show that inter-individual variation of genetic ancestry within populations is the rule rather than the exception. Since anthropometric phenotype is expected to vary as a function of genetic history rather than subjective social notions of population affiliation, it is clear why individual genetic ancestry admixture must be measured in order to properly infer such phenotypes. These results also highlight the differences between social notions of “race” and genetic ancestry.

Statistical Error

Bias in admixture estimation is one source of “error”, and is caused by the continuous nature and error in estimating parental allele frequencies. Fortunately, this source of error is easily quantified. Since the alleles are not strictly private to each group, it is expected there will exist a certain level of statistical “noise” inherent to the practice of estimating admixture using these alleles. This error is expected to be higher for smaller and/or less informative panels of AIMs. To estimate the bias inherent to the 176 AIM panel and ML method, we performed simulation studies. Genotypes for samples from one population were created in-silico, BGA estimated and the level of outside-group admixture taken as an estimate of the bias. Table 4a shows the computed BGA for simulated ancestral samples. Mean BGA estimated for each group showed >95% affiliation with the expected population and established the limits of BGA expected from non contributing populations that may be expected in an individual when using these AIMs and the ML method.

TABLE 4a
175 of Genomes Best AIMs

| | | | | | | Total |
|---|---|---|---|---|---|---|
| | AFR | EUR | EAS | NAM | Total | Admixture Avg. |
| Africans | 98.21 | 0.93 | 0.71 | 0.15 | 100 | 1.79 |
| Europeans | 0.4 | 96.36 | 1.5 | 1.74 | 100 | 3.64 |
| East Asians | 0.08 | 1.43 | 95.48 | 3.01 | 100 | 4.52 |
| Native Americans | 0 | 1.16 | 2.08 | 96.76 | 100 | 3.24 |
| | | | | | | Avg. 3.30 |

Threshold of affiliation by percentages for samples of polarized, binary affiliation, above which results indicate fractional affiliation with a p ≦ 0.05 using the 171 admixture test.

As can be seen in this table, the bias in affiliation for one parental group in one population is not necessarily the same as for another in that population, or in different populations. The population bias for all ancestral groups was less than 5% in this sample, indicating that levels of individual (not population) admixture less than 5% are generally not reliable, though the ancestry bias varies from population to population and admixture type to admixture type. For example, the average Sub-Saharan African sample shows 0.93% European admixture as a function of bias but the average East Asian sample shows 3.01% Indigenous American admixture due to bias (TABLE 4a). Levels of admixture above which there is a 95% certainty of bona-fide affiliation, not caused by bias, were calculated and range from the low single digit percents to 12.5% depending on the population and admixture and the values were noted to generally reflect the genetic distances between the groups (TABLE 4b).

TABLE 4b
Levels of admixture above which there is a 95% certainty
of bona-fide affiliation.

| | AFR | EUR | EAS | NAM |
|---|---|---|---|---|
| Africans | <3.0% | 7% | 5% | <3.0% |
| Europeans | 3.50% | <3.0% | 9% | 10% |
| East Asians | 0 < 3.0% | 8% | <3.0% | 12.50% |
| Native | <3.0% | 7.5% | 11.50% | <3.0% |
| Americans | | | | |

This work indicates that IGAA estimates using the panel of AIMs described herein are accurate to within a few percentage points. That is, an individual may be determined to be 90% European/10% East Asian, and if this determination is wrong the correct answer is most likely a similar set of percentages such as 95% European/5% East Asian, as opposed to a vastly different answer such as 50% European/50% East Asian. A variety of other validation exercises have been carried out—to quantify repeatability, concordance of admixture proportions in family pedigrees, in genealogies, and the like.

Indirect Inference of Various Clinical Phenotypes

The primary reason clinicians want to know about ancestry is to assist them in reconstructing a clinical phenotype; that is, to enable generalizations about phenotypes. Clearly, certain physical characteristics are unevenly distributed among the worlds various peoples, such as skin pigmentation levels, hair and eye colors, and even aspects of facial features such as eye shape, soft facial tissue morphology and cranial features. Of course many clinical phenotypes are equally disparate—including but not limited to those of drug metabolism and response. Inferring phenotype from ancestry is an example of an indirect inference, since we rely on measurements of AIMs and not the phenotypically functional loci that underlie the expression of the trait when making such predictions (i.e., neutral SNPs). In this case, we use ancestry as a proxy for the net effect of the character of these loci in individuals, a character which is distributed to a certain extent as a function of ancestry. Making an inference of physical trait value from measurement of the functional loci themselves is an example of direct inference, since we rely on measurements of the locus (presumably gene) variants that cause trait characters. However, to practice direct inference, genetic research must obviously have identified the genes that cause the trait. Unfortunately, this has only been accomplished satisfactorily so for iris color (by DNAPrint laboratories, and Frudakis et al., 2003, supra) and to a lesser extent, variable skin pigmentation. For all other traits, genetic inference must be made indirectly. Many clinical traits will prove to be of such complex genetics, involving such a large number of genes and variants that genetic research cannot practically be carried out to “solve” them in a direct sense. For example, if response to drug X is a function of 8 different interacting genes, each with a equal and modest affect on expression of the response phenotype, sample sizes in the hundreds of thousands would be needed to detect their associations. Clearly, for many clinical traits, the indirect method of inference is the only one that will be practicable for the foreseeable future.

Although the human population is relatively undifferentiated compared to other species, we know from our everyday experiences that various human subpopulations exhibit certain distinct, characteristic physical qualities called anthropometric phenotypes or divergent traits. Most anthropometric phenotypes are known to be superficial in nature, contributing only towards outward appearance but the expression of many disease phenotypes carry a strong ethnic component and many authors have implemented approaches based on measurements of genetic ancestry admixture to map their genetic determinants (Fernandez et al., Obes Res (2003) 11:904-911; Molokhia et al., Hum Gene (2003) 112:310-318; McKeigue P, Am J Hum Genet (1997) 60:188-196; Zhu et al., Nat Genet (2005) 37:177-181; Reiner et al., Am J Hum Genet (2005) 76:463-477). Indeed, variation in xenobiotic metabolism genes explain most all pharmacokinetic variation and therefore a large part of inter-individual variation in drug response, and it is known that ethnic variation in xenobiotic metabolism gene sequences are extreme (Frudakis et al., 2001). To infer anthropometric or clinical traits using genetic ancestry admixture we must use a method that is empirical and database-driven rather than theoretical. For example, high skin melanin content is a polyphyletic trait—meaning the trait is exhibited among populations that correspond to a variety of human phylogenetic lineages (i.e. Papauans, West Africans, aboriginal Australians, South Asian Indians). For example, MC1R variants are known to exist, and have been identified by various investigators (See, e.g., Tables 5a), including expected frequencies for observed phenotypes (Table 5b).

TABLE 5a
Frequency of MC1R variants.

| | | | | Palmer | Flanagan | | Bastiaens | Kennedy | Duffy |
|---|---|---|---|---|---|---|---|---|---|
| MC1R | Freq | Box et | Smith et | et al., | et al., | Box et | et al., | et al. | et al., |
| Variant | (5) | al., 1997 | al., 1998 | 2000 | 2000 | al., 2001 | 2001 | 2001 | 2004 |
| Val60Leu | 12.4 | NA | NA | NA | Partial | NA | NA | OR = 2.3 | OR = 6.4 |
| | | | | | Recessive | | | (1.0-5.4) | (2.8-14.9) |
| Asp84Glu | 1.1 | — | — | — | Recessive | — | p < 0.004 | OR = 5.1 | OR = 62.8 |
| | | | | | with | | | (1.4-18.3) | (17.6-223.7) |
| | | | | | RHC | | | | |
| | | | | | allele | | | | |
| Val92Met | 9.7 | NA | NA | NT | NA | NA | NA | NA | OR = 5.3 |
| | | | | | | | | | (2.2-12.9) |
| Arg142His | 0.9 | — | NT | NT | Recessive | — | p < 0.0001 | OR = 49.2 | — |
| | | | | | | | | (16.8-145.5) | |
| Arg151Cys | 11.1 | p < 0.001 | p < 0.0015 | p < 0.01 | Recessive | p < 0.0001 | p < 0.0001 | OR = 20.7 | OR = 118.3 |
| | | | | | | | | (11.5-37.3) | (51.5-271.7) |
| Arg160Trp | 7.1 | p > 0.05 | p < 0.001 | p < 0.001 | Recessive | p < 0.0001 | p < 0.0001 | OR = 12.5 | OR = 50.5 |
| | | | | | | | | (7.1-21.9) | (22.0-115.8) |
| Arg163Gln | 5 | — | — | NT | NA | NA | NA | NA | OR = 2.4 |
| | | | | | | | | | (0.5-11.3) |
| His260Pro | NT | NT | NT | NT | NT | NT | p < 0.008 | OR = 9.9 | NT |
| | | | | | | | | 9.9 (2.6-37.0) | |
| Asp294His | 2.8 | p < 0.05 | p < 0.005 | p < 0.001 | Recessive | p < 0.0001 | p < 0.0001 | OR = 12.4 | OR = 94.1 |
| | | | | | | | | (3.8-40.5) | (33.7-263.1) |

NA = not associated.
NT = not tested.
— = insufficient sample size for association.

TABLE 5b
Phenotypes associated with MC1R variants.

| MC1R | | Expected | Observed | Observed |
|---|---|---|---|---|
| genotype | No. | % red | No. Red | % red |
| “+/+” | 425 | 0 | 0 | 0 |
| “r/+” | 412 | 0.2 | 4 | 1 |
| “R/+” | 344 | 2.5 | 5 | 1.5 |
| “r/r” | 111 | 1 | 1 | 0.9 |
| “R/r” | 203 | 11.8 | 22 | 10.8 |
| “R/R” | 73 | 62.1 | 49 | 67.1 |
| Total | 1568 | | 81 | |

So while it may be true that normally pigmented West Africans tend to have a skin melanin (M) index values above 35, it is not true that all individuals with a skin melanin index value above 35 are of West African origin or even of partial origin. Traits that owe their origin to the forces of natural selection, such as xenobiotic metabolism traits, hair color and iris color are frequently found in many different lineages due to convergent or parallel evolution. To properly infer such traits using genetic ancestry, rather than the selective forces as determinative variables, we must use a method that accommodates natural genetic variation within populations, varying environmental factors and polyphylogeny and consider that most “answers” will be in the form of ranges representing the diversity of values found in a population of a given type of admixture, rather than discrete values. If a sample is typed as 100% East Asian, the clinician cannot objectively determine whether this person is likely to respond to a drug differently than the population of Europeans assessed in the clinical trials of the drug. The statement “the individual should respond like most East Asians” is meaningless to a scientist, because it is very difficult to define the average East Asian response in objective terminology.

The way around these problems is to build databases of response to the drug for individuals from around the world, determine the IGAA of each sample, and query the database with an IGAA result and its confidence interval. If the IGAA result is indicative of a particular response phenotype, individuals returned from such a query would show a consistent value for this phenotype—a value that is different from those with significantly different IGAA results.

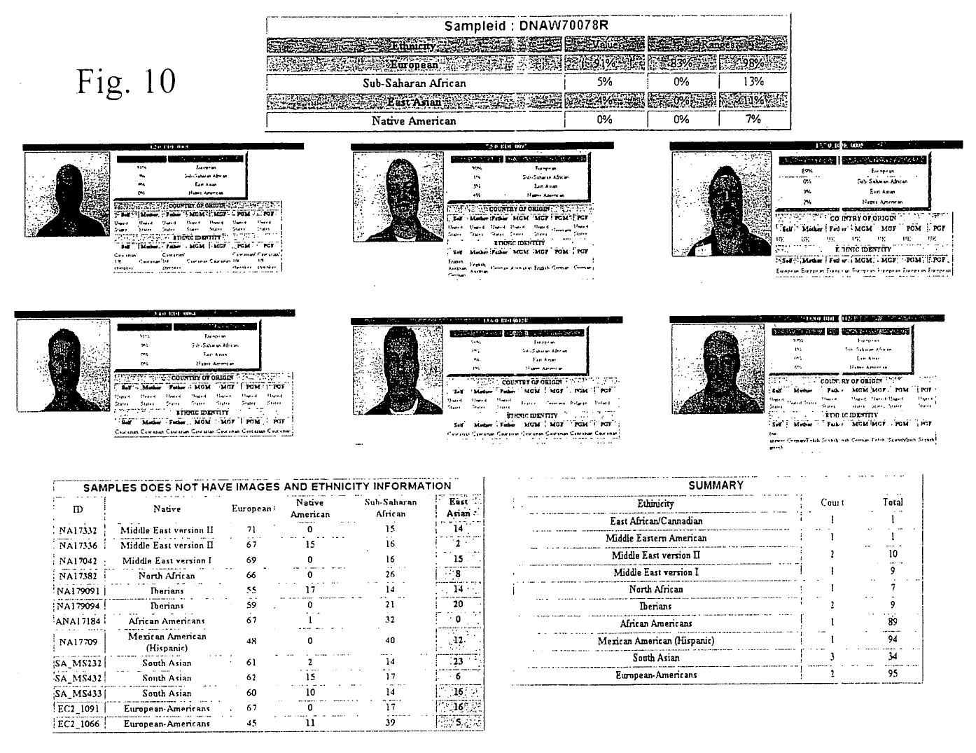

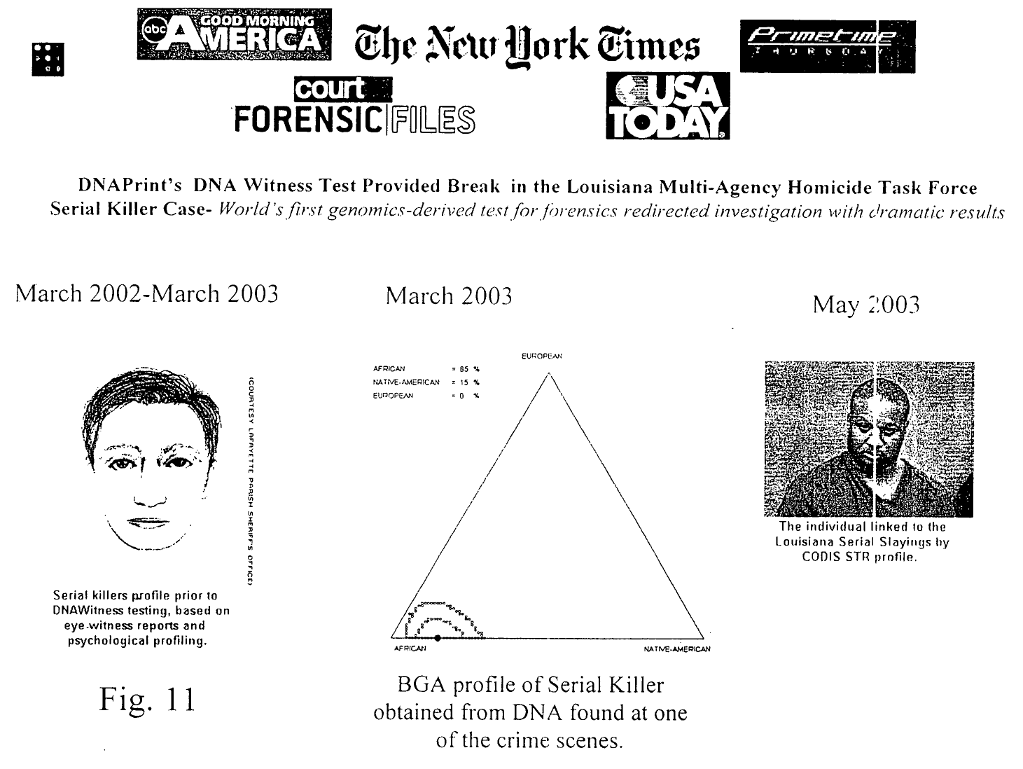

We have applied this same logic and methodology within the field of forensics. For example, if an investigator learned that the DNA found at a murder scene was donated by an individual showing 70% European and 30% West African ancestry, he or she would not know precisely what this tells them about skin and hair color unless he looked at a large collection of individuals of similar proportions, summarized the statistics relevant to the expression of the trait in this subgroup, and compared these statistics to subpopulations for other types of BGA profiles. The conclusion would not simply be that a person of 70% European and 30% West African ancestry has “dark” skin, but rather that this person has skin of a melanin index value 400 or above, which is very different than the value that would apply for the average European American with, say, <5% African ancestry. DNAPrint has constructed one such database with IGAA estimates obtained from the 176 AIMs discussed herein. FIG. 9 shows how skin melanin index value correlates with sub-Saharan African (AF) admixture for samples within this database. Generally, higher AF values are meaningful indicators of higher M values, and the correlation is strong enough that we can make reasonably accurate inference of M value given the AF value. FIG. 10 shows an example of a return from querying the database with the results 83%-98% European/0-13% AF/0-11% East Asian/0-7% Indigenous or Native American. Six individuals returned are shown as representative of the larger return—and all of them appear outwardly as “Caucasian” phenotype. A software program can measure the average M value, inter-ocular distance, hair color—any phenotype including clinical traits—and we can test whether this value is significantly different from other IGAA returns. Using this approach we can learn which phenotype values are indicated by a given IGAA result and which are not, and we can construct our physical and/or clinical description using these phenotypes only. A computer program could construct the description, using the specific values and ranges indicated by the database return for these phenotypes, and using generic (non-specific) values for other phenotypes. In this way, a computer could construct the “artists rendering” of a forensics suspect or an in-silico inference of likely response to a particular drug for a given patient based on genomic information rather than subjective human reports. We can take this a step further and query the database for “self-descriptors” such as “race” or self-reported “response” and learn what the person is most likely to describe him/herself as (FIG. 10 bottom left) or what type of response the person is likely to report—in patient rather than physician terms. We could also query for continent or subcontinent of origin and learn the possible origins for person who donated a crime scene sample (FIG. 10 bottom right). Some of these methods were applied to help resolve the Louisiana Serial Killer case in 2003. Two separate eye-witness reports had indicated a “Caucasian” suspect, but over a years worth of expensive DNA dragnets had failed to find a match. DNAPrint analyzed a sample common to several of the rape/murder scenes and determined the donor was a person of 85% sub-Saharan African/15% Native or Indigenous American mix, and more importantly, likely to express a darker skin shade with other overtly African characteristics (FIG. 11). Approximately 1.5 months later, the suspect was apprehended, based in large part on the genomic profile provided, which helped focus investigative effort and redirect the investigation away from unscientific forms of evidence. This case is a good example of the power that testable, repeatable, objective and quantifiable genomic science has to offer the field of forensics, which has been burdened until recently with largely subjective, non-falsifiable forms of evidence.

Identification of Samples Using Infrared Analysis of the Iris

It is known that changing infrared LED light sources causes a corresponding change in the spectral reflection of the iris from the cornea. This can be further manipulated to detect a contact lens which might contain a printed fake iris pattern riding on the spherical surface of the cornea, rather that in an internal plane within the eye.

The eye color data can be handled by treating the categories as nominal and analyzing the three dichotomous variables, namely, blue/grey versus other, green/hazel versus other, and non-blue versus other. Alternatively, eye color can be considered as a continuum, in that color is largely a function of quantitative differences in the density and size of melanosomes, and possibly the quantity or quality of melanin within the melanosomes.

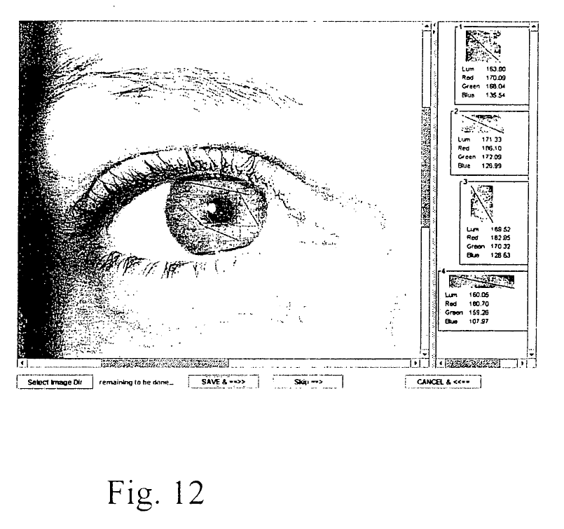

An example of a graphic interface image for analyzing eye color is shown in FIG. 12. Boxes are drawn in each of four quadrants of the iris, and average pixel luminosity, red reflectance, green reflectance, and blue reflectance determined for each box. Downstream software computes an average among these four boxes to produce four values for each iris that are expected to uniquely represent the melanin content of the iris relatively independent of distribution pattern.

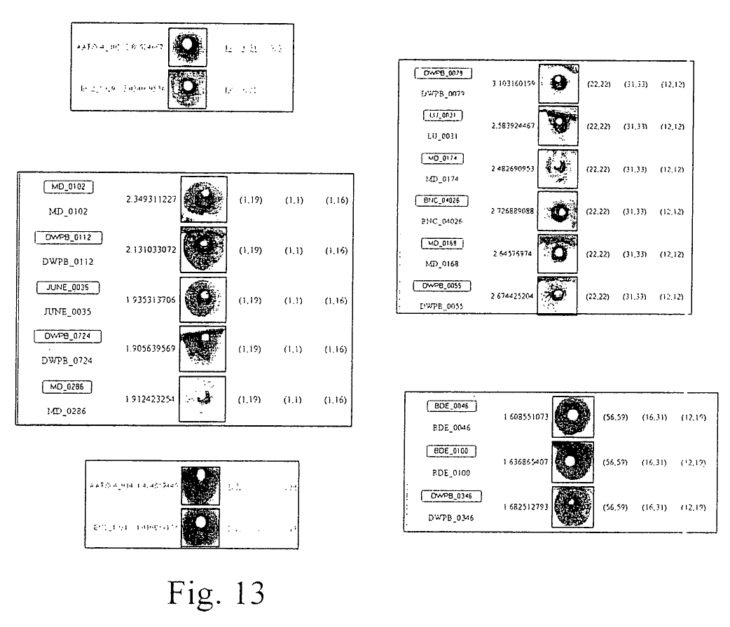

As shown in FIG. 13, each box represents a unique multilocus OCA2 genotype. The Iris Melanin Index for each iris is shown in the second column of each box and a digital photograph of the iris is shown in the third column. For the multilocus OCA2 genotype to be the same for each sample, the values in these four boxes must be identical. Information may be obtained on phasing of haplotypes among these four regions. For these samples, the concordance observed is significant-even among irises of mixed blue/brown color.

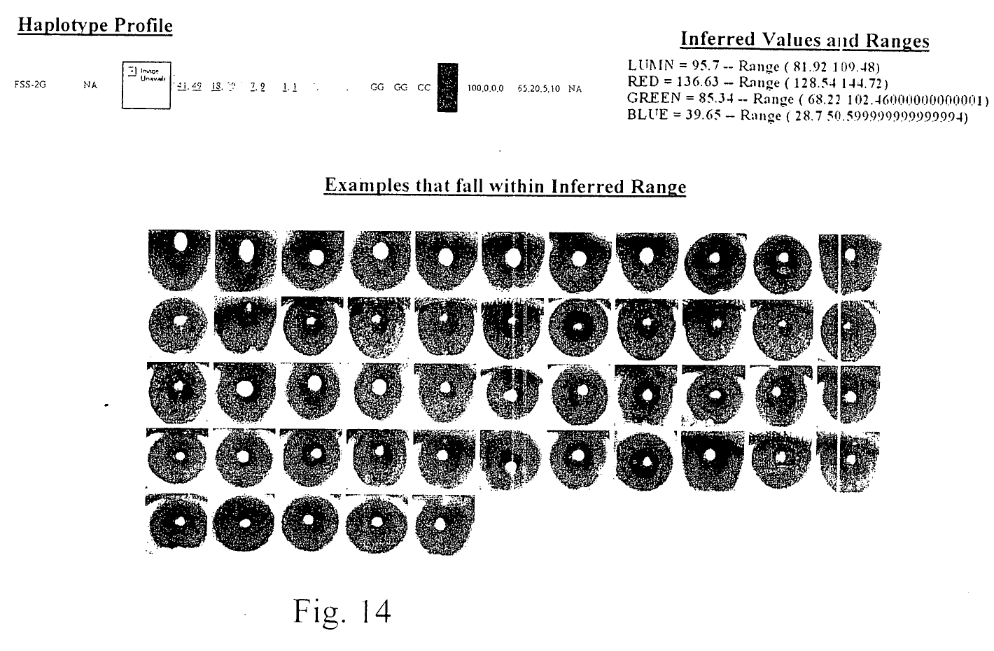

An infrared scan can be performed on a test individual, and based on examples that fall within the infrared range, the test individual can be identified (see, e.g., FIG. 14). The inference shown in FIG. 14 was performed using a hierarchical method. This iris was predicted to be of a range of colors falling in the middle of the color spectrum, and the prediction was correct. Further verification may obtained by comparisons between other biometric data, including bit not limited to, fingerprints, facial recognition, and the like.

It should be understood that various changes can be made in the function and arrangement of elements without departing from the scope of the invention as set forth in the appended claims and the legal equivalents thereof.

What is claimed is:

1. A system for identifying and tracking a sample from an individual, the system comprising:
a sample container;
an individual's identification sample portion coupled to the sample container; and
means for identifying the individual's identification sample portion.

2. The system of claim 1, wherein the individual's identification sample portion is configured to accept a fingerprint, human iris scan, human eye color, facial recognition photo, DNA, or a combination thereof.

3. The system of claim 2, wherein the means for identifying the individual's identification sample is an instrument.

4. A method of using a system for identifying and tracking a sample from an individual, the system comprising:
placing the sample from the individual in a sample container configured to hold the sample;
obtaining and placing an individual's identification sample on an individual's identification sample portion coupled to the sample container; and
identifying the individual's identification sample using appropriate instruments to identify the sample.

5. The method of claim 4, wherein the individual's identification sample portion is configured to accept a fingerprint, human iris scan, human eye color, facial recognition photo, DNA, or a combination thereof.

6. A method of identifying or tracking a sample from an individual comprising:
i) a user interacting with a graphic interface to enter data associated with the individual and inputting biometric data, wherein the data is selected from the group consisting of DNA data, physical characteristics, and medical information, or a combination thereof;
ii) sending the biometric data to a database comprising authentic biometric data previously obtained from the individual;
iii) processing the biometric data to compare the inputted biometric data with corresponding authentic biometric data; and
iv) verifying the identity of the individual if the inputted biometric data meets a minimal criteria when compared with the authentic biometric data.

7. The method of claim 6, wherein the physical characteristics are selected from a photograph of the individual, a fingerprint of the individual, or an eye scan of the individual.

8. The method of claim 6, further comprising:
v) placing a DNA sample in a container;
vi) attaching a label which annotates the sample based on inputted biometric data; and
vii) associating the sample with a data file, wherein the data file comprises biometric data selected from the group consisting of physical characteristics and medical information, or a combination thereof.

9. The method of claim 6, wherein the medical information is updated.

10. The method of claim 9, wherein an alert is generated when personal information or medical status of the individual changes.

11. The method of claim 8, wherein the threshold for identification for a DNA sample comprises:
a) contacting the DNA sample of the individual with hybridizing oligonucleotides,
wherein the hybridizing nucleotides can detect nucleotide occurrences of single nucleotide polymorphisms (SNPs) of a panel of at least about ten ancestry informative markers (AIMs) indicative of a population structure correlated with the trait, and
wherein the contacting is performed under conditions suitable for detecting the nucleotide occurrences of the AIMs of the test individual by the hybridizing oligonucleotides; and
b) identifying, with a predetermined level of confidence, a population structure that correlates with the nucleotide occurrences of the AIMs in the test individual, wherein the population structure correlates with a trait.

12. The method of claim 11, wherein the panel comprises at least about twenty AIMs.

13. The method of claim 11, wherein the trait comprises biogeographical ancestry (BGA).

14. The method of claim 11, wherein at least one AIM of the panel is not linked to a gene linked to the trait.

15. The method of claim 13, wherein the BGA comprises a proportion of a sub-Saharan African, Native American, IndoEuropean, or East Asian ancestral group, or a combination of the ancestral groups.

16. The method of claim 15, wherein the BGA comprises a proportion of at least three ancestral groups.

17. The method of claim 13, wherein the BGA comprises proportions of at least sub-Saharan African and IndoEuropean ancestral groups; Native American and IndoEuropean ancestral groups; East Asian and Native American ancestral groups; or IndoEuropean and East Asian ancestral groups.

18. The method of claim 13, wherein the BGA comprises proportions of at least Native American, East Asian, and IndoEuropean ancestral groups; or sub-Saharan African, Native American, and IndoEuropean ancestral groups.

19. The method of claim 11, further comprising identifying, with a predetermined level of confidence, a sub-population structure of the population structure that correlates with the nucleotide occurrences of the AIMs in the test individual, wherein the sub-population structure correlates with a trait.

20. The method of claim 11, wherein the hybridizing oligonucleotides comprise oligonucleotide primers, the method further comprising contacting the sample with a polymerase, under condition suitable for generation of a primer extension product, wherein determining the nucleotide occurrence of a SNP comprises detecting the presence of the primer extension product.
